(19) United States
(12) Patent Application Publication
(10) Pub. No.: US 2013/0071367 A1
(43) Pub. Date: Mar. 21, 2013

(54) ANIMAL FEED FOR CALVES FOR CONDITIONING THE INTESTINAL FLORA

(71) Applicants: Johann Bauer, Ismaning (DE); Karin Schwaiger, Moosburg (DE)

(72) Inventors: Johann Bauer, Ismaning (DE); Karin Schwaiger, Moosburg (DE)

(73) Assignee: Technische Universitat Munchen, Munchen (DE)

(21) Appl. No.: 13/638,031

(22) PCT Filed: Mar. 28, 2011
(86) PCT No.: PCT/EP2011/054743

§ 371 Date: Dec. 3, 2012

(30) Foreign Application Priority Data
Mar. 29, 2010 (DE) 10 2010 013 209.8

(51) Int. Cl. A61K 35/74

(52) U.S. Cl. CPC A61K 35/741
USPC 424/93.44

(57) ABSTRACT

The present invention is directed to a animal feed for calves comprising calf-specific bacteria, optionally lipids, proteins, vitamins and/or mineral materials for conditioning of the intestinal flora in calves, and thus, for prophylaxis and/or treatment of diarrhea in calves.

The present invention is directed to an animal feed for calves comprising calf-specific bacteria like microorganisms of the genera Lactococcus, Lactobacillus, Leuconostoc Enterococcus, Streptococcus, Propionibacterium, Bifidobacterium, Eubacterium, Pediococcus, Veillonella, Bacteroides and/or Escherichia, and optionally lipids, proteins, vitamins and/or mineral materials for conditioning of the intestinal flora of calves, and therefore for prophylaxis and/or treatment of diarrhea in calves. The animal feed according to the present invention in particular allows the conditioning of newborn calves' intestinal flora, in particular within the first 24 to 48 h after the calves' birth.

In Germany, about 5 million calves are born per year, whereof still more than 10% of all newborn calves perish within the first half year despite intensive therapeutic measures in individual therapy and flock-prophylactic measures, respectively. About 75% of losses in calves are traced back to infectious diseases (DLZ, 2006; Bothmer and Budde, 1992). Therein, diarrhea plays an important role and is on top, with about 80% of infection-determined losses.

Most notably, diarrheas within the first days of life constitute a particular severe problem worldwide: About 60% of losses occur in the first and an additional 30% within the second week of life (Pohlenz et al., 1978; Elze et al., 1994; Kohara et al., 1997). Calves having perished due to diarrhea cause an economic damage of about [symbol] 31.5 million per year in Germany. However, economic damages caused by the death of a calf are by far higher due to feed costs, wage costs and veterinarian costs. This calculation does not yet comprise costs caused by surviving diarrhea-affected calves which require treatment. Providing that about 20% of all living-born calves come down with diarrhea (Metz and Metz, 1984), further damages of [symbol] 58.2 million occur in Germany based on medical costs of [symbol] 60 per diseased calf. Further subsequent damages which are difficult to be calculated, accrue via reduced breeding progress, diminished breeding selection options and reduced performance in adolescent or even adult cattle, as frequency and severity of calf disease strongly correlate to the later performance as dairy cow: adolescent animals which did not or only one time require veterinarian treatment during growing show a significant lower first birth age and a higher lactation performance than calves which required treatment. The importance of calf's health becomes in particular evident in view of the useful life: Of those calves being healthy during growing, only about 20% go off during first lactation, while the respective percentage of frequently treated calves accounts for 60% (Trilk and Münch, 2004). Further, the necessary additional animal purchase bears the risk of germ introduction which is not to be underestimated (Brändle, 2006).

Calves suffering from strong diarrhea may loose up to 20% of their body mass in liquid. Along with said liquid electrolytes such as sodium, potassium, chloride and hydrogen carbonate are lost. Finally, following dehydration, acidosis and hypoglycemia the general condition is rapidly worsened extending to so called “downer calves”. Thus, early liquid and electrolyte substitution as well as coverage of the nutritional requirement are considered the most important measures. This is achieved by intravenous infusion in severe cases such as in insufficiently drinking calves or “downer calves”. Non-invasive oral rehydration therapy is preferred as long as the calf can drink by itself (Rademacher et al., 2002), for what a multitude of electrolyte potions are available, whose composition is based on mineral materials and easy available carbohydrates, partly in combination with astringent or mucosa saving additives such as spruce needle extract, citrus marc, pectin or psyllium (e.g. Enerlyte/Virbac; Glutellac/Bayer; Diätmix/Bewital; Medolyt/VetroStar MS; Diakur plus/Boehringer Ingelheim, Effydral/Essex; Floracid Novo/Albrecht). Administration of antibiotics to eliminate pathogens is recommended only if a complication or an additional disease (e.g. navel inflammation or pneumonia) is prevalent as the physiological intestinal flora is destroyed by this causal therapy, which may lead later on to a bacterial dysbiosis (Mansfeld et al., 2005). Taking restrictions into consideration, which apply for animals in food production, various allaying agents are available such as adsorbents (alumium sicilacte, carbo medicinalis), astringents (e.g. tannin or tanninic acid), or motility inhibitors, whose application is, however, prohibited in food producing animals. Additionally, non steroidal anti-inflammatory drugs (NSAID) can be employed for an anti-inflammatory effect (e.g. acetylic salicylic acid, ketoprofen), which, however, just serve as symptomatic therapy like all aforementioned anti-diarrhea agents (Rosa Liste, “Rose list”, 2009).

Timely and sufficient first milking care is considered the most important factor in protection from intestinal infections. Vaccination of the dam prior to birth can further improve the colostrum's protective effect as the produced antibodies cross over to the milk and protect the calf from infections, which in turn prevents and reduces pathogen reproduction and excretion, respectively. Thereby, in combination with optimized keeping and hygiene conditions, infection stress on newborn calves should be reduced (Mansfeld et al., 2005). Probiotics are increasingly administered orally to support as well as to normalize the intestinal flora. These feed additives consist of living microorganisms which survive the craw's acidic environment in sufficient numbers to exercise a health promoting effect in the intestine. Such microorganisms are mostly, lactic acid bacteria like Lactobacillus, Enterococcus and Bifidobacterium species. These microorganisms, which also belong to the physiologic intestinal flora, are considered to act preventively against diarrhea as pathogen growth-inhibiting competitive germs in addition to further positive characteristics (such as e.g. vitamin production, lactose disintegration or improvement of mineral material resorption) (Newbold, 1995). In addition, prebiotics such as non-digestible oligosaccharides and oligofructides are frequently used which serve as sole nutrition for probiotic microorganisms to selectively support the microorganisms. To date, only bifidogenic oligosaccharides such as inulin and its hydrolysis product oligofructose and galactooligosaccharides comply with these criteria. Various commercially available products against claves' diarrhea are supplemented with probiotics. Floracid novo (Albrecht) or Mega Bac (Mega Sprint) contain e.g. Enterococcus faecium strain “Cernelle 68” at a concentration of almost 10¹⁰ CFU/kg. Although diverse studies showed a certain positive effect of individual strains in calves (Beeman, 1985; Bonaldi et al. 1986; Umburger et al., 1989), losses caused by calves' diarrhea still remain a serious problem. Herein it is to be considered that the probiotic strains were originally not intended for specific employment in calves. For example, “Cernelle 68” was isolated from the human intestine. As the intestinal flora exhibits significant differences between various animal species and humans, related employment of arbitrarily chosen microorganisms not originating from the homologous animal species has to be evaluated critically.

The problem underlying the present invention was the development of an animal feed, which is specifically balanced for calves' intestinal flora, and allows to prevent and/or treat diarrhea in calves.

This problem is solved by the subject matter of claim 1. Moreover, the present invention comprises the production of the animal feed as well as its use in the prevention and/or treatment of diarrhea in calves.

The great advantage of the present invention is that the animal feed is specially balanced for calves and their specific intestinal flora, e.g., calf-specific combinations of different species of gram positive and negative bacteria for use in conditioning the intestinal flora of newborn calves. The dense colonization of the intestine's inner wall by microorganisms, in particular bacteria which are at first missing in newborn mammals, is designated an intestine flora. The calf's intestinal flora consists of and comprises, respectively, different species of gram positive and gram negative bacteria, whose appearance and composition is typical for the calf's intestinal flora, as well as the amount of different bacteria and bacteria species, respectively, related to the total amount per g of feces. Rumen and colon are the organs with the largest and most comprehensive population of microorganisms in a ruminant animal, wherein microorganisms comprise in general protozoa or metazoa, and bacteria, in particular gram positive and negative bacteria, fungi and algae. In the colon, a total amount of germs up to 10¹¹ CFU per g feces can be reached (Dowd et al., 2008). According to doctrine, intestinal colonization of the calf starts immediately after birth (Braegger, 2004; Nicolet, 1985). Colonization of the newborn first occurs via the oral cavity by germs of the birth channel, the immediate maternal environment, the mammary gland, the dam's feces and the germ species accidentally occurring in the immediate proximity of the newborn (Ducluzeau, 1983; Isik, 2004). This first colon colonization takes place very quickly, however, large differences are encountered: “sterile” samples of meconium, the newborn's first feces, exist as well as samples containing up to 10⁸ CFU per g meconium. About 48 h after birth, mammals reach a maximum germ load of 10⁸ CFU per g (Ducluzeau, 1983; Jimenez et al., 2008). However, the newborn's intestinal flora differs significantly from the one of grown-up mammals. Primary settlers are mostly aerobic microorganisms which serve as “environment preparers” for subsequent colonization of anaerobic germs. Anaerobic microorganisms finally dominate in grown-up mammals, and aerobic microorganisms account for just 10% of the intestinal flora (Isik, 2004). The newborn's intestinal flora is very instable and numerous dominant colonies disappear after some days or are substituted by others (Favier et al., 2003, Lukas et al., 2007). Administration of colostrums, i.e. the administration of the dam's first milk, further nutrition with milk exchangers, stable hygiene, environmental germ flora and further management factors perform a strong influence on the development of the intestinal flora (Pfirrmann and Böhm, 2000). The following Table 1 shows germ groups of grown-up cattle's intestinal flora.

TABLE 1

| | |
|---|---|
| Main flora (>90%) | Bifidobacteria |
| | Lactobacilli |
| | Bacteroides spp. |
| Concomitant flora (<1%) | E. coli |
| | Enterococci |
| | Propionibacteria |
| Remainder flora (<0.01%) | Clostridia |
| | Proteus |
| | Staphylococci |
| | Pseudomonadaceae |

Calf's bacterial diarrhea pathogens are, e.g., E. coli, Salmonella spp., Clostridium perfringens and Campylobacter spp., respectively. E coli belongs to the normal intestine inhabitants and only a part of its serotypes causes infectious diseases. These serotypes differ in so-called virulence factors from “harmless” E. coli. Virulence factors provide the ability to connect to intestinal cells and to produce certain toxins (Kaske and Kunz, 2003). E. coli often appears as secondary pathogen after Rota or Cornea virus infections and worsens the clinical picture (Bothmer and Budde, 1992). In newborn calves, diseases appear especially in the first two weeks of life. In the beginning, the thin feces is of yellow color, later it turns aqueous, interstratified with unmetabolized clotted milk compounds (Bothmer and Budde, 1992). The cause is considered to be strong colonization of the intestinal mucosa by E. coli with late or insufficient administration of colostrum. After oral take-up, Salmonella spp. migrate into the small intestine. Due to massive reproduction, they lead to an inflammation in the intestine associated with bad smelling, at first yellowish, later grey-greenish and finally dark brown to black diarrhea and temperature. Most calves die within a few days due to fluid loss or due to bacteria migrating via the intestinal mucosa into the blood, leading to central nervous disorders in connection with palsy. Salmonella infections may occur in cattle of every age in principle, but calves are most sensitive due to their not yet fully developed immune system, wherein an epidemic-like course occurs frequently. The few surviving calves often permanently excrete (“permanent eliminators”) these microorganisms, and thus, constitute a threat to the whole farm. In numbers, Salmonella by far play a less important role than E. coli in calves' diarrhea—but due to their zoonosis-like character as well as the gravity of their progressions, Salmonella constitute a prominent diarrhea pathogen. Clostridium perfringens can be isolated from the feces of calves without clinically findings of the calf, and in addition, is known as etiologic factor in enteric toxicema (Rycke et al., 1986). Herein developing small intestine necroses are manifested in acute, severe, sometimes bloody diarrhea and heavy pain symptoms (Kaske and Kunz, 2003). Campylobacter spp. are considered likewise as zoonosis pathogens and can be detected both in the feces of calves without clinical findings as well as in diarrhea-affected calves (Meylan, 2007). It is assumed that pathogens of older permanent eliminators without clinical findings are passed to newborn claves (Höfle, 2006). Upon section, an intense colonization of the colon is detectable; besides mucoid feces, ill calves do not exhibit clear symptoms of disease (Höfle, 2006).

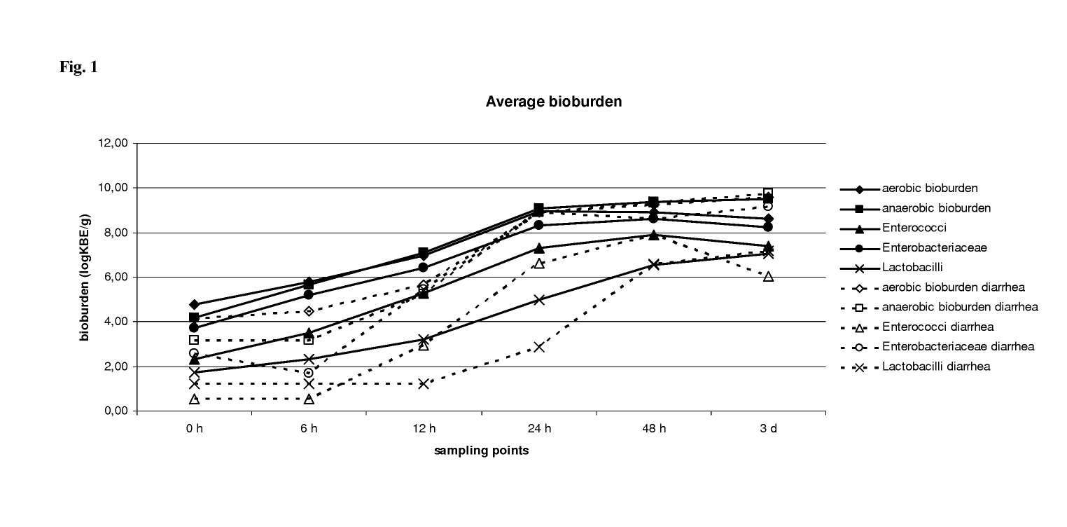

An examination of calves' intestine flora showed that the intestine flora of healthy and of diarrhea-affected calves exhibits differences in a qualitative and quantitative aspect already before the occurrence of the diarrhea, i.e. before the occurrence of clinical symptoms, as demonstrated in FIG. 1. These differences are particularly significant in particular within the first 24 to 48 h after a calf's birth. Some microorganisms, in particular bacteria, are missing or reduced in number in the intestinal flora of diarrhea-affected calves or of those that will suffer from diarrhea. These are in particular Enterococci and/or Lactobacilli. It was surprisingly found out that the intestinal flora of calves suffering from diarrhea primarily does not exhibit an excessive reproduction of pathogenic microorganisms, in particular bacteria, but rather that diarrhea is primarily caused by the absence or decline of protective microorganisms. The absence of microorganisms strongly manifests in particular in the first 24 to 48 h after the calf's birth so that this time frame is of high importance for conditioning the calf's intestinal flora and thus, for prevention and/or the treatment of a calf.

Here, the animal feed according to the invention counteracts and prevents and/or balances the imbalance of the calf's intestinal flora. The animal feed preferably comprises or consists of, respectively, one or more microorganisms, which are particularly preferred gram positive and/or negative bacteria. Optionally, the animal feed comprises proteins, lipids, vitamins and/or mineral materials. The animal feed is fed to the calf preferably directly after birth, in particular in the first hours, the first days and/or weeks after birth. The animal feed according to the invention is administered preferably in the first 1 to 6 months of life, particular preferably in the first 1 to 3 months of life. In a very preferred embodiment, the animal feed according to the invention is administered within the first 24 to 48 h after the calf's birth.

In another embodiment, the animal feed for calves is fed to cattle of grown-up age.

In a further preferred embodiment, the animal feed is administered prenatally to the calf via the dam. In a further preferred embodiment the animal feed is administered to the calf prenatally and in the first 1 to 2 weeks, or 24 to 48 h after birth directly to the calf. The animal feed of the present invention is added as dietary supplement to standard calves or cattle feed, or is fed to calves or cattle as sole nutrition. Particularly advantageous, the animal feed results in the conditioning of the intestinal flora in still unborn or newborn calves, respectively, whereby a possible intestinal flora's imbalance of newborn claves is balanced in view of the composition, i.e. in view of the different species of microorganisms, and/or in view of the amount of individual microorganisms of different species in relation to the total amount of microorganisms. In particular, an imbalance in intestinal bacteria such as Enterococci and/or Lactobacilli frequently appears, wherein the imbalance is within the species and/or amount of intestinal flora-building bacteria. The present animal feed or dietary supplement, respectively, enables the building of a physiologic intestinal flora typical for healthy calves. Therefore, the use of the animal feed of the invention preferably results in the prophylaxis of diarrhea in calves, but also the treatment of diarrhea in calves or cattle of grown-up age.

The present invention is not limited to an animal feed for calves. In fact, the present invention comprises animal feeds and dietary supplements, respectively, for all mammals, in particular for humans, horses, pigs, dogs and cats, whereby the animal feed and the dietary supplement, respectively, comprise characteristic microorganisms typical for the specific animal in amounts typical and characteristic for the mammal.

In addition to oral administration of typical microorganisms via an animal feed or a dietary supplement, respectively, the present invention further comprises e.g. intravenous, rectal or intravaginal administration by the administration of the microorganisms e.g. in form of tablets, in particular foam tablets, solutions, gels, or suppositories.

Alternatively or in addition to the oral administration of the calf-specific microorganisms of the invention via an animal feed, the microorganisms comprised by the animal feed are preferably administered rectally or intravaginally, e.g. by means of suppositories, tablets, in particular foam tablets or foam. Microorganisms are blended preferably with one or more pharmaceutical carrier substances for preparation of the rectal or intravaginal dosage form. Carrier substances are for example binders, disintegrates, surfactants, adsorbing promoters, wetting agents, adsorbing agents, lubricants, foaming agents, fillers, extenders, humectants and similar diluents or excipients, aseptic agents, coloring agents, antioxidant agents or preservers. In a further embodiment, the rectal or intravaginal dosage form is supplied with an antibiotic. The rectal administration of the microorganisms to the calf preferably occurs in particular within the first 24 to 48 h after the calf's birth.

In case of prenatal prophylaxis of the calf, microorganisms are administered to the dam preferably intravaginally, e.g. 1 to 10 days prior to birth, preferably 1 to 5 days prior to birth, in particular preferably 1 to 2 days prior to birth.

Gram positive bacteria present in the animal feed are preferably selected from the group consisting of the genera Lactococcus, Lactobacillus, Leuconostoc Enterococcus, Streptococcus, Propionibacterium, Bifidobacterium, Eubacterium, Pediococcus and Clostridium. Preferred gram negative bacteria present in the animal feed are selected from the group consisting of the genera Escherichia, Pseudomonas, Veillonella, Bacteroides and Proteus.

Particularly preferred bacteria are bacteria selected from the group consisting of Lactococcus chungangensis, Lactococcus lactis subsp. cremoris, Lactococcus lactis subsp. diacetilactis, Lactococcus lactis subsp. horidae, Lactococcus lactis subsp. lactis, Lactococcus piscium, Lactococcus plantarum, Lactococcus raffinolactis, Lactobacillus acidophilus, Lactobacillus helveticus, Lactobacillus casei, Lactobacillus delbrueckii, Lactobacillus plantarum, Lactobacillus parabuchneri, Lactobacillus ferintoshensis, Lactobacillus salivarius, Enterococcus faecium, Enterococcous faecalis, Enterococcus avium, Enterococcus casseliflavus, Enterococcus durans, Enterococcus gallinarum, Enterococcus hirae, Enterococcus lactis, Enterococcus malodoratus, Enterococcus mundtii, Enterococcus raffinosus, Enterococcus pseudoavium, Enterococcus cecorum, Enterococcus columbae, Enterococcus saccharolyticus, Enterococcus dispar, Enterococcus sulfureus, Enterococcus asini, Enterococcus villorum, Enterococcus haemoperoxidus, Enterococcus moraviensis, Enterococcus ratti, Enterococcus pallens, Enterococcus gilvus, Enterococcus seriolocida, Enterococcus solitarius, Enterococcus flavescens, Escherischa coli, Bifidobacterium adolescentis, Bifidobacterium animals subs. animalis, Bifidobacterium animalis subsp. lactis, Bifidobacterium bifidum, Bifidobacterium breve, Bifidobacterium infantis, Bifidobacterium lactis, Bifidobacterium longum, Bifidobacterium ruminantium, Pediococcus acidilactici, Pediococcus cellicola, Pediococcus claussenii, Pediococcus damnosus, Pediococcus ethanolidurans, Pediococcus inopinatus, Pediococcus parvulus and Pediococcus pentosaceus.

In one embodiment, the animal feed comprises additionally an adsorbent, an astringent, inulin and/or a motility inhibitor.

The intestinal flora of healthy calves is still only low characterized and depends amongst others on the animals' age. The content of anaerobicly growing microorganisms accounts for about 10¹⁰ CFU/g, the amount of aerobic microorganisms for about 10⁸ CFU in healthy calves and grown-up cattle. Lactobacilli similarly like Enterococci account for about 10⁶-10⁷ CFU/g, whereby the part of Entercocci is slightly lower (10⁵-10⁷ CFU/g) in healthy calves and grown-up cattle. From about the third day after birth onwards, the calf's intestinal flora becomes more stabile and converges more and more to the intestinal flora of a grown-up cattle. The first 24 to 48 h after the calf's birth are in particular essential for the intestinal flora's conditioning in cattle.

In addition, the present invention is directed to the production of the animal feed of the invention, which comprises calf-specific microorganisms of the genera Lactococcus, Lactobacillus, Leuconostoc, Enterococcus, Streptococcus, Propionibacterium, Bifidobacterium, Eubacterium, Pediococcus, Veillonella, Bacteroides and/or Escherichia and optionally lipids, proteins, vitamins and/or mineral materials. The components of the animal feed are blended in the ratios desired for the individual and particular needs.

The invention is further directed to the production of tablets, in particular foam tablets, solutions, gels or suppositories comprising the microorganisms, wherein the components are blended in the ratio desired for the individual and particular needs.

In another embodiment, the present invention refers to a method of preventing and/or treating mammals, in particular calves, with the animal feed and/or a tablet, in particular a foam tablet, solution, gel or suppository comprising the microorganisms of the animal feed.

The following examples describe the investigation of the calves' intestinal flora, whereby the present invention is not limited to the embodiments described in the examples.

### EXAMPLE 1

### Investigation of the Intestinal Flora of Calves

Calf feces from about 150 to 200 calves are taken rectally in aseptic conditions in definite, short intervals of a few hours in particular within the first seven days of life, starting with the meconium sample. Additionally, a colostrum sample is obtained in each case. The feces samples are taken according to the following scheme: 0 h, 6 h, 12 h, 24 h, 48 h, 3 d and 7 d after birth. Thereby, 7 samples are gained from each calf within the first week of life. Additionally, information on the calf s identity, gender, race, date of birth, birth weight, weight development, medication, particularities and the like was recorded. The state of health was recorded beyond the sampling period.

All samples were bacteriologically investigated, both qualitatively and quantitatively. Different selection enrichment methods guarantee the comprehensive overview on the prevalent germ diversity. The aerobic and the anaerobic mesophilic total germ count as well as Enterobacteriaceae (including E. coli and Salmonella) Campylobacter, Enterococci and Lactobacilli are determined. The germ content is determined according to the surface-spatula-method on the basis of Gedek (1974). Therefore, samples are streaked onto the following culture media after preparation of a dilution row and are counted out after incubation for 48 h at 37° C.:
Blood agar (aerobic mesophilic total germ count)
Schaedler agar (anaerobic mesophilic total germ count)
Gassner agar (Enterobacteriaceae)
CATC agar (Enterococci)
LAMVAB agar (Lactobacilli)

Additionally, Salmonella and Campylobacter, respectively, are selectively enriched according to DIN EN 12824 and on the basis of the method of the Bavarian state agency for health and food safety.

A section of the investigation is depictured in FIG. 1. Evaluation of 24 calves shows a significantly reduced germ load of Enterococci and Lactobacilli within the first 48 h, in particular within the first 24 h after the calves' births. Total germ count of aerobic microorganisms in healthy calves (♦) and calves which came down with diarrhea within 14 days post partum (⋄), the total germ count of anaerobic microorganisms in healthy calves (▪) and in calves which came down with diarrhea (Δ), the count of Enterococci in healthy calves (▴) and in calves which came down with diarrhea within 14 days post partum. (Δ), the count of Enterobacteriaceae in healthy claves () and in calves which came down with diarrhea within 14 days post partum (○), as well as the count of Lactobacilli in healthy calves (-x-) and in calves which came down with diarrhea within 14 days post partum (--x--) were determined.

### EXAMPLE 2

### Diarrhea Prophylaxis

The development of the intestinal flora in calves with or without diarrhea is retrospectively compared and statistically evaluated. Additionally, data on potential influencing factors are integrated into the analysis, e.g. feeding of the dams, feeding of the calves, colostrum intake, flock health, mode of keeping (ecological/conventional; tied/open land keeping; individual keeping/group keeping), season, therapies and the like.

Evaluation of differences in the bacterial biodiversity in healthy and diarrhea-affected calves results in conclusions related to the protective effect of different microorganism collectives. Based on these findings, candidate germs, in particular Laktobazilli, Enterococci, Bifidobacteria, Veillonella, Bakteroides and/or E. coli, are selected for production of an animal feed and orally administered to newborn calves. Untreated or placebo-treated calves of the same farms served as controls. State of health and development of the bacterial intestinal flora is likewise recorded for placebo-treated calves.

1. Animal feed for calves comprising calf-specific microorganisms of the genera Lactococcus, Lactobacillus, Leuconostoc, Enterococcus, Streptococcus, Propionibacterium, Bifidobacterium, Eubacterium, Pediococcus, Veillonella, Bacteroides and/or Escherichia as well as optionally lipids, proteins, vitamins and/or mineral materials.

2. Animal feed according to claim 1, wherein the Lactobacillus is selected from the group consisting of Lactobacillus acidophilus, Lactobacillus helveticus, Lactobacillus casei, Lactobacillus delbrueckii, Lactobacillus plantarum, Lactobacillus parabuchneri, Lactobacillus ferintoshensis, and/or Lactobacillus salivarius.

3. Animal feed according to claim 1, wherein the Enterococcus is selected from the group consisting of Enterococcus faecium, Enterococcous faecalis, Enterococcus avium, Enterococcus casseliflavus, Enterococcus durans, Enterococcus gallinarum, Enterococcus hirae, Enterococcus lactis, Enterococcus malodoratus, Enterococcus mundtii, Enterococcus raffinosus, Enterococcus pseudoavium, Enterococcus cecorum, Enterococcus columbae, Enterococcus saccharolyticus, Enterococcus dispar, Enterococcus sulfureus, Enterococcus asini, Enterococcus villorum, Enterococcus haemoperoxidus, Enterococcus moraviensis, Enterococcus ratti, Enterococcus pallens, Enterococcus gilvus, Enterococcus seriolocida, Enterococcus solitarius, and/or Enterococcus flavescens.

4. Animal feed according to claim 1, wherein the Bifidobacterium is selected from the group consisting of Bifidobacterium adolescentis, Bifidobacterium animals subs. animalis, Bifidobacterium animalis subsp. lactis, Bifidobacterium bifidum, Bifidobacterium breve, Bifidobacterium infantis, Bifidobacterium lactis, Bifidobacterium longum, and/or Bifidobacterium ruminantium.

5. Animal feed according to claim 1, wherein the Pediococcus is selected from the group consisting of Pediococcus acidilactici, Pediococcus cellicola, Pediococcus claussenii, Pediococcus damnosus, Pediococcus ethanolidurans, Pediococcus inopinatus, Pediococcus parvulus and/or Pediococcus pentosaceus.

6. Animal feed according to claim 1, wherein Lactococcus is selected from the group consisting of Lactococcus chungangensis, Lactococcus lactis subsp. cremoris, Lactococcus lactis subsp. diacetilactis, Lactococcus lactis subsp. horidae, Lactococcus lactis subsp. lactis, Lactococcus piscium, Lactococcus plantarum and/or Lactococcus raffinolactis.

7. Animal feed according to claim 1, wherein Escherichia is Escherichia coli.

8. Animal feed according to claim 1, wherein the animal feed additionally comprises an astringent, an adsorbent, inulin and/or a motility inhibitor.

9. Use of the animal feed according to claim 1 for conditioning the intestinal flora of calves for prophylaxis and/or treatment of diarrhea.

10. Use of the animal feed according to claim 9, wherein the animal feed is administered in the first 1 to 12 months of the calves' life, preferably in the first 1 to 6 months, in particular preferably in the first 1 to 3 months in order to condition the new-born calf's intestinal flora.

11. Use of the animal feed according to claim 9, wherein the animal feed is administered within the first 24 to 48 h after the calf's birth.

12. Use of the animal feed according to claim 9, wherein the microorganisms of the animal feed are administered to the calf prenatally by administering the animal feed to the dam.

13. Use of the animal feed according to claim 9, wherein the calf-specific microorganisms are administered rectally.

14. Method for producing the animal feed for calves according to claim 1, wherein the calf-specific microorganisms of the genera Lactococcus, Lactobacillus, Leuconostoc, Enterococcus, Streptococcus, Propionibacterium, Bifidobacterium, Eubacterium, Pediococcus, Veillonella, Bacteroides and/or Escherichia are blended with a lipid, a protein, a vitamin and/or a mineral material.

15. Method for production of a rectal dosage form of the calf-specific microorganisms based on the animal feed according to claim 1, wherein the calf-specific microorganisms of the genera Lactococcus, Lactobacillus, Leuconostoc, Enterococcus, Streptococcus, Propionibacterium, Bifidobacterium, Eubacterium, Pediococcus, Veillonella, Bacteroides and/or Escherichia are blended with one or more pharmaceutical carrier substances.

16. Method according to claim 15, wherein the pharmaceutical carriers are selected from the group consisting of binders, disintegrates, surfactants, adsorbing promoters, wetting agents, adsorbing agents, lubricants, foaming agent, fillers, extenders, humectants and similar diluents or excipients, septic agents, coloring agents, antioxidant agents or preservers.
